(19) United States
(12) Patent Application Publication
(10) Pub. No.: US 2003/0228374 A1
(43) Pub. Date: Dec. 11, 2003

(54) Topical treatment for skin irritation

(76) Inventor: Thomas C. Pesacreta, Lafayette, LA (US)

Correspondence Address: William W. Stagg, Attorney-at-Law, Durio, McGoffin & Stagg, P.O. Box 51308, Lafayette, LA, 70505-1308, US

(21) Appl. No.: 10/164,349

(22) Filed: Jun. 7, 2002

(51) Int. Cl. A61K 33/42; A61K 33/00

(52) U.S. Cl. 424/601; 424/715; 424/717

(57) ABSTRACT

The present invention comprises a method and composition for treating skin disorders such as seborrheic dermatitis of the scalp, dandruff, and psoriasis. The method includes topically applying to the skin a solution containing a treatment composition comprising one or more types of alkaline buffers such as bicarbonate, carbonate, dibasic phosphate, and tribasic phosphate having a pH in the range of about 7.1 to about 10.8 and a cation such as potassium or sodium serving as a counter ion. The treatment composition is delivered in a pharmaceutically acceptable vehicle at concentrations that vary according to both the severity of the skin disorder, the buffer vehicle, and the ingredients themselves. This biologically benign treatment has been found to efficiently and very quickly treat several types of skin disorders, including but not limited to the redness, dandruff and itching that can occur following skin cleansing with soap.

### FIELD OF THE INVENTION

The present invention is generally related to the treatment of skin irritations and more particularly to the topical treatment of seborrheic dermatitis, dandruff, and psoriasis or similar skin conditions with an alkaline buffer solution.

### BACKGROUND OF THE INVENTION

Many patents have been granted that purport to address the treatment of a group of skin ailments that include seborrheic dermatitis, dandruff, and some types of psoriasis such as scalp psoriasis. The link between these diseases is that they have several symptoms in common including skin loss (i.e. flaking), itching and, especially in the case of scalp psoriasis and seborrheic dermatitis, redness of the skin. Although significant time and research has been directed toward curing these ailments only limited success has been achieved. The symptoms of seborrheic dermatitis, dandruff, or psoriasis continue to represent a significant daily psychological and physiological irritation for millions of people, and a significant market for pharmaceutical companies.

Part of the reason that treatments have been ineffective is that the causes of these disorders are unknown. Psoriasis has been correlated with the presence of certain types of bacteria. Seborrheic dermatitis and dandruff have been correlated with the presence of fungi. However, in none of these cases has the cause of the symptoms been unequivocally established. While some treatments are effective for some individuals, other individuals have chronic symptoms that are resistant to treatment.

All the abovementioned diseases affect skin cells that, if unaffected by disease, would normally undergo a multi-week cycle of growth and subsequent necrosis. Another commonality between seborrheic dermatitis, dandruff, and some types of psoriasis is that the lifetime of the afflicted skin cells is drastically reduced, leading to precocious flaking in a few days or less. In an attempt to extend the lifetime of these cells, anti-dandruff and medicated shampoos have been utilized that contain a wide array of chemical agents. These agents are designed to have increased effectiveness if they are retained on the skin where they can act to retard the growth of fungi and microorganisms. Many of these agents such as coal tar, selenium, and zinc pyrithione, can have negative effects on mammalian cells if they are used at inappropriate concentrations or for extended periods and the manufacturer's instructions on such agents can include a warning that a physician should be consulted if the product is used for an extended period. Given the possible risks of using such chemicals for long periods and the chronic symptoms that continue to occur in some individuals who have used such agents, the use of new, biologically benign treatments has become an attractive alternative. In addition, the abovementioned agents are expensive.

Several patents for the treatment of epidermal inflamation with relatively benign chemicals have recently been granted. For the prevention of mast cell degranulation Perricone (U.S. Pat. No. 6,051,244) recommended the topical application of fructose 1-6 diphosphate, in combination with either sodium or potassium as a counter ion. This chemical which is a naturally occurring substance and an intermediate in the glycolytic pathway in all mammalian cells is proposed as a cure for inflamation of epidermal tissues, especially mucosal inflamation. Katz (U.S. Pat. No. 5,952,384) claimed the efficacy of pyruvate, another common metabolic intermediate of mammalian cells, for the treatment of inflamation due to the production of hydrogen peroxide in lung tissues. Hornack (U.S. Pat. No. 6,063,406) claimed a complex composition composed of cation-free water, calcium chloride, linear long-chain polyphosphate, sodium bicarbonate, and cross-linked polyacrylamide was useful for the treatment of skin disorders. Hornack specified the use of the composition at a pH of 3.5-4.0. At this pH the bicarbonate will have been converted to either carbonic acid or carbon dioxide. Finally, another treatment involving the use of the potassium ion (K+) was suggested by Oge (U.S. Pat. No. 5,955,067) as an effective topical treatment of acne vulgaris, psoriasis, and seborrhea.

The need for a diverse array of skin treatments may be because the factors that lead to skin inflamation or itching, and the resultant loss of skin cells are varied. Nevertheless, a commonality of most conventional treatments for dandruff, seborrheic dermatitis, and psoriasis is that they involve the use of some type of cleansing agent such as an ionic surfactant (i.e. soap, the primary active ingredient of which is, in many cases, sodium dodecyl sulfate also known as SDS, or sodium lauryl sulfate). This cleansing agent generally kills or counteracts pathogens such as bacteria and fungi, and may remove chemicals produced by the host tissue that, in some cases, contribute to the symptoms of the skin disorder.

Unfortunately in many cases, especially where the symptoms are chronic, the area of the skin that requires cleansing has become irritated and is highly susceptible to chemical irritants. It is therefore paradoxical that a surfactant such as SDS, which itself is also a known skin irritant, is a significant ingredient in virtually all soaps. SDS is a commonly used agent to provoke skin irritation for experimental research (Lee and Maibach, 1995). SDS can cause a disorganization of lipid bilayers (e.g. cell membranes), and subsequent hyperhydration of the stratum corneum (Leveque et al. 1993). This leads to transepidermal water loss (TEWL) and increases blood flow that is clinically visible as erythema (Van der Valk et al., 1984). Thus, skin flaking due to dessication and redess can be caused by application of SDS. It is also known that a pre-existing skin irritation can pre-dispose the skin to an even higher sensitivity to SDS (Frosch and Kligman, 1977; and Murahata et al., 1986). Thus, treatment with soap can be a problematic factor when used on irritated skin. Substantial research has been and is currently being conducted by the American medical community to reduce the irritative effects of soap (Frosch and Kligman, 1979; Zhai et al., 1999; Zhai et al., 2000; Gabard et al., 2001).

It is the purpose of the present invention to provide an ameliorative treatment to counteract the symptoms of dandruff, seborrheic dermatitis, and psoriasis. This ameliorative treatment may act directly upon the biotic factors that provoke the symptoms of the above mentioned skin conditions or, without prejudice to the invention, it may act upon irritation, itching and skin cell loss as it may be provoked by the irritative side effects of cleansing agents such as soap.

### SUMMARY OF THE INVENTION

The present invention provides a composition and method for the treatment of epidermal disorders. It presents a water-soluble alkaline topical treatment solution or paste that is effective for the reduction and/or prevention of symptoms of seborrheic dermatitis, dandruff, and psoriasis. The solution or paste that can be used either alone or in combination with other commonly used hair treatments such as, but not being limited to, soap, conditioner, or rinse that may be applied periodically as the symptoms require. The proposed composition and method is physiologically benign and inexpensive so that its use will be facilitated for those with chronic symptoms. Other features of the present invention will become clear from the following description when read in conjunction with the appended claims.

### DETAILED DESCRIPTION OF EMBODIMENT

In general, the treatment method of the present invention involves the topical application to affected sites of a composition comprised of an aqueous alkaline solution or paste selected from the group comprised of the anions bicarbonate, carbonate, dibasic phosphate, tribasic phosphate and having a pH value in the range of about 7.1 to about 10.8. Combined with the alkaline buffer will be a pharmaceutically acceptable monovalent or divalent cation such as but not limited to sodium, potassium, or lithium the purpose of which is to serve as counterions and therefore facilitate the formation of an alkaline buffer solution or paste. The chemicals described above may be dissolved in water, or may be used in combination with a pharmaceutically acceptable vehicle such as shampoo, conditioner or rinse.

The bicarbonate, carbonate, dibasic phosphate, and tribasic phosphate suggested for the alkaline solution or paste may be used singularly or in combination with one another and are to be supplied as a salt in combination with the pharmaceutically acceptable cation, such as sodium or potassium that also may be used singularly or in combination with one another. As used herein, a “salt” includes compounds having one or more cations complexed with bicarbonate, carbonate, dibasic phosphate, or tribasic phosphate.

The concentration of the proposed compositions required to bring about a reduction of skin inflammation and skin flaking is dependent upon the severity and the extent of the symptoms as they occur on each individual. Concentrations of the proposed compositions as low as about 0.3% (weight to volume) applied once a week has been found to be effective for treatment of relatively mild symptoms. Concentrations of about 1.0% to about 3.0% applied daily to the afflicted area have been found effective for moderate cases of skin inflammation and flaking. For areas of the skin that exhibit severe symptoms, or when the extent of the skin disorder is confined to a very limited area, the daily topical application of a concentrated paste of a composition comprising sodium bicarbonate or dibasic potassium phosphate plus a small amount of water has been found to be effective.

A final consideration with regard to the concentration of ingredients is their effect on the pH of the dissolving solution being used in combination with the alkaline salt ingredients. The pH of distilled water can differ dramatically from the pH of ordinary tap water depending upon its source. Strongly acidic water will therefore require higher concentrations of bicarbonate, carbonate, dibasic phosphate, tribasic phosphate for effectiveness. Further, the pH of the alkaline solution may be simply that of the alkaline salt solution, or it may be adjusted within the stated alkaline range by the addition of a commonly available alkaline reagent such as sodium hydroxide (NaOH) or acid such as hydrogen chloride (HCl).

The method of treatment is also related to the severity of the symptoms of each individual. For mild cases the ingredients may be used directly along with a pharmaceutically acceptable vehicle such as a shampoo, rinse or conditioner. For more difficult cases it is advisable to dissolve the ingredients in an aqueous rinse that is applied to the afflicted area immediately after skin cleansing. Then the ingredients are thoroughly rubbed into the afflicted area of the skin. The ingredients may be left on the skin for a few minutes and then rinsed off with water, or they may be left on the skin. For the most severe symptoms, salts of either bicarbonate or dibasic phosphate can be made into a paste with water and gently rubbed onto the afflicted area. This paste is allowed to stay on the skin for a few minutes and then is rinsed off.

### EXAMPLE 1

As an illustrative example, a treatment composition and method of applicant's invention was prepared as composition having the following components and quantities. First, 5.0 gm of sodium bicarbonate was added to a mixing vessel. Then, 5.0 ml of tap water (pH 8.0) was added to the mixing vessel and the components were then mixed to produce an alkaline, paste-like composition. The patient had a locally inflamed scalp and flaking skin associated with seborrheic dermatitis on the forehead, and had recently cleansed the skin with shampoo. The paste-like composition was rubbed on to the skin of a test patient to form a thin layer over the inflamed area. The paste-like composition remained in place on the test patient for 5.0 minutes and then was rinsed from the surface of the skin with tap water. As the hair dried, the scalp inflammation and skin flaking on the test patient showed marked improvement in the next 0.5 hours after application of applicant's composition.

### EXAMPLE 2

As another illustrative example of applicant's invention, a composition having the following components and quantities was prepared as follows. First, 1.0 gm of potassium bicarbonate was added to a mixing vessel. Then, 100.0 ml of distilled water as a solvent was added to the mixing vessel and the components were then mixed to produce a suspended solution having a pH value in the range of about 8.6 to about 9.0. The resulting solution was rubbed onto the freshly cleansed scalp of a test patient having flaking skin associated with dandruff. After allowing the resulting solution to remain on the scalp of the test patient for about one-half hour, the patient's scalp was thoroughly rinsed with tap water. As the patient's scalp dried, the skin flaking on the test patient showed marked improvement.

### EXAMPLE 3

As another illustrative example, the treatment composition and method of applicant's invention was prepared as composition having the following components and quantities. First, 3.0 gm of sodium carbonate was added to a mixing vessel. Then, 100.0 ml of tap water was added to the mixing vessel and the components were then mixed to produce a solution composition having a pH value in the range of about 9.8 to about 10.8 which was adjusted to pH 9.0 with 1.0 N hydrogen chloride (HCl). The resulting solution was then used as a post cleansing rinse and rubbed on to the skin of a test patient having a severely inflamed scalp and flaking skin associated with scalp psoriasis to form a thin layer over the infected area. The solution remained in place on the test patient for about 2.0 minutes and then was rinsed from the surface of the skin with tap water. The scalp inflammation and skin flaking on the test patient showed marked improvement in the next one-half hour after application of applicant's solution composition.

### EXAMPLE 4

As still another illustrative example of the treatment composition and method of applicant's invention, a composition having the following components and quantities was prepared. First, a salt comprising 0.1 gm of sodium carbonate, 0.5 gm of potassium carbonate, and 1.0 gm of dibasic sodium phosphate were added to a mixing vessel. Then, 100.0 ml of distilled water was to the mixing vessel and the components were then mixed to produce a solution having a pH value in the range of about 7.5 to about 8.0. The resulting solution was then rubbed on to the skin of a test patient having an inflamed scalp and flaking skin associated with seborrheic dermatitis to form a thin layer over the infected area. The paste-like composition remained in place on the test patient for about 10 minutes and then was rinsed from the surface of the skin with heated tap water. The scalp inflammation and skin flaking on the test patient showed marked improvement in the next one-half hour after application of applicant's composition.

### EXAMPLE 5

As still another illustrative example of the treatment composition and method of applicant's invention, a composition was prepared having the following components and quantities. First, a salt comprising 5.0 gm of sodium bicarbonate was added to a mixing bottle having an equal weight of a generally available shampoo. The components were then mixed to produce a solution having a pH value in the range of about 8.6 to about 8.8. The resulting solution was then rubbed on to the skin of a test patient having an inflamed scalp and flaking skin associated with seborrheic dermatitis to form a frothy layer over the infected area. The composition remained in place on the test patient for about 5 minutes and then was rinsed from the surface of the skin with heated tap water. The scalp inflammation and skin flaking on the test patient showed marked improvement in the next one-half hour after application of applicant's composition.

It has been observed that applicant's proposed solution and treatment method is capable of dramatically reducing the incidence of symptoms related to skin disorders such as dandruff, seborrheic dermatitis and psoriasis. It has been observed, without prejudice to the present invention, that these effects can occur during the period that follows skin cleansing. It has also been observed that these effects can occur in individuals from a wide variety of genetic backgrounds including male, female, Caucasian, and African, and in age groups from 25 to 52 years of age. It has also been observed that, in the case of chronic sufferers, the duration of cessation of symptoms can vary from a period of twenty-four hours to one week or longer.

The present application describes several compositions and techniques, which have been shown to be effective for treating the symptoms of skin disorders that include skin redness, flaking and itching. Although it is not clearly understood how these symptoms can be prevented by the treatment composition according to the present invention, it is believed that this invention is effective for the treatment of these symptoms as they are provoked during skin cleansing procedures with soaps, or as they may occur after skin cleansing. The proposed compositions can be used either by themselves in solution with water or other pharmaceutically acceptable vehicles, or in combination either with each other, or in combination with shampoos, hair rinses, conditioners or other pharmaceutically acceptable vehicles.

The above description is intended for teaching the ordinary person with skill in the art how to practice the present invention, but it is not intended to present in detail all the obvious modifications and variations of it which will become apparent to the skilled worker upon reading the description. It is intended, that all such obvious modifications and variations be included within the scope of the present invention, which is defined by the following claims. The claims are intended to cover the claimed components and steps in any sequence that is effective to meet the intended objectives, unless otherwise indicated.

I claim:

1. A composition for the topical treatment of skin, comprising:
a) an alkaline buffer solution comprising anions selected from the group consisting of bicarbonate, carbonate, dibasic phosphate, and tribasic phosphate;
b) a pharmaceutically acceptable cation, and;
c) a pharmaceutically acceptable vehicle for holding said alkaline buffer solution and said cation in solution.

2. The composition for the topical treatment of skin as recited in claim 1, wherein the concentration of said alkaline buffer solution by weight to volume is within the range of about 0.3% to about 10.0%.

3. The composition for the topical treatment of skin as recited in claim 1, wherein said cation is selected from the group consisting of sodium and potassium.

4. A composition for the topical treatment of skin as recited in claim 2, wherein said cation is selected from the group consisting of sodium and potassium.

5. A composition for the topical treatment of skin, comprising:
a) an alkaline buffer having a pH value in the range of about 7.1 to about 10.8selected from the group consisting of bicarbonate, carbonate, dibasic phosphate, and tribasic phosphate;
b) a pharmaceutically acceptable cation; and
c) a pharmaceutically acceptable vehicle for holding said alkaline buffer and said cation in solution.

6. The composition for the topical treatment of skin as recited in claim 5, wherein said alkaline buffer has a concentration by weight to volume within the range of about 0.3% to about 10.0%.

7. The composition for the topical treatment of skin as recited in claim 5, wherein said cation is selected from the group consisting of sodium and potassium.

8. The composition for the topical treatment of skin as recited in claim 6, wherein said cation is selected from the group consisting of sodium and potassium.

9. A composition for the topical treatment of skin, comprising:
a) an alkaline buffer having a pH value in the range of about 7.1 to about 10.8 selected from the group consisting of bicarbonate, carbonate, dibasic phosphate, and tribasic phosphate;
b) a pharmaceutically acceptable cation;
c) a reagent to adjust the pH of the solution as desired within the range of about 7.1 to about 10.8, and;
d) a pharmaceutically acceptable vehicle for holding said alkaline buffer, said cation, and said reagent in solution.

10. The composition for the topical treatment of skin as recited in claim 9, wherein the concentration of said alkaline buffer solution by weight to volume is within the range of about 0.3% to about 10.0%.

11. The composition for the topical treatment of skin as recited in claim 9, wherein said cation is selected from the group consisting of sodium and potassium.

12. The composition for the topical treatment of skin as recited in claim 10, wherein said cation is selected from the group consisting of sodium and potassium.

13. The composition for the topical treatment of skin as recited in claim 9, wherein the concentration of said alkaline buffer solution by weight to volume is in the range of about 10.0% to about 75.0%.

14. The composition for the topical treatment of skin as recited in claim 9, wherein said cation is selected from the group consisting of sodium and potassium.

15. The composition for the topical treatment of skin as recited in claim 10, wherein said cation is selected from the group consisting of sodium and potassium.

16. The composition for the topical treatment of skin as recited in claim 9, wherein said pharmaceutically acceptable vehicle is selected from the group consisting of hair rinses, hair conditioners, and shampoos.

17. A composition for the topical treatment of skin, comprising:
a) an alkaline buffer selected from the group consisting of bicarbonate and dibasic phosphate;
b) a pharmaceutically acceptable cation; and
c) a vehicle for combining said alkaline buffer and said cation into a paste.

18. The composition for the topical treatment of skin as recited in claim 17 wherein the pH value of said paste is in the range of about 8.6 to about 8.8.

19. A method for the topical treatment of skin, comprising the steps of:
a) providing an alkaline buffer having a pH value in the range of about 7.1 to about 10.8, said alkaline buffer selected from the group consisting of bicarbonate, carbonate, dibasic phosphate, and tribasic phosphate;
b) providing a pharmaceutically acceptable cation;
c) dissolving said alkaline buffer and said cation in a pharmaceutically acceptable suspending vehicle to produce a suspended solution;
d) rubbing said selected area of skin with said suspended solution;
e) allowing said solution to remain on said selected area of skin for at least about one minute; and
f) rinsing said selected area of skin with water.

20. The method as recited in claim 19 comprising the additional step of cleaning said selected area of skin prior to the step of rubbing said selected area of skin with said suspended solution.

21. The method as recited in claim 19 wherein said alkaline buffer in said suspended solution has a concentration by weight to volume within the range of about 0.3% to about 10.0%.

22. The method as recited in claim 19 wherein, said pharmaceutically acceptable cation is selected from the group consisting of sodium and potassium.

23. The method as recited in claim 20 wherein, said alkaline buffer in said suspended solution has a concentration by weight to volume within the range of about 0.3% to about 10.0%.

24. The method as recited in claim 23 wherein, said pharmaceutically acceptable cation is selected from the group consisting of sodium and potassium.

25. The method as recited in claim 21 wherein, said pharmaceutically acceptable suspending vehicle is in the form of a shampoo.

26. The method as recited in claim 22 wherein, said pharmaceutically acceptable suspending vehicle is in the form of a shampoo.

27. The method as recited in claim 22 wherein, said alkaline buffer in said suspended solution has a concentration by weight to volume within the range of about 0.3% to about 10.0%.

28. The method as recited in claim 27 wherein, said step of providing an alkaline buffer having a pH in the range of about 7.1 to about 10.8 includes adjusting said pH to the desired value with a solution of hydrogen chloride.

29. The method as recited in claim 27 wherein said suspended solution is a paste.

30. The method as recited in claim 28 wherein, said pharmaceutically acceptable suspending vehicle is selected from the group consisting of shampoos, hair rinses and hair conditioners.

31. A composition for the topical treatment of skin consisting essentially of the following:
a) about 0.3 percent to about 10.0 percent by weight of an alkaline buffer selected from the group consisting of sodium carbonate, potassium carbonate and dibasic sodium phosphate;
b) vehicle means for holding said alkaline buffer in solution; and
c) an acidic pH adjusting substance in an amount sufficient to adjust the pH of said composition to a value in the range of about 7.1 to about 10.8.

32. The composition for the topical treatment of skin as recited in claim 31, further comprising a pharmaceutically acceptable cation.

33. The composition for the topical treatment of skin as recited in claim 32 wherein, said an acidic pH adjusting substance is a solution in water of hydrogen chloride.

34. The composition for the topical treatment of skin as recited in claim 33 wherein, said vehicle means for holding said alkaline buffer in solution is water in an amount sufficient to form said composition into a paste.

35. The method as recited in claim 33 wherein, said vehicle means for holding said alkaline buffer in solution is selected from the group consisting of shampoos, hair rinses and hair conditioners.
